(19) United States
(12) Patent Application Publication
(10) Pub. No.: US 2011/0111002 A1
(43) Pub. Date: May 12, 2011

(54) TRANSPORT AND DELIVERY OF GLUTATHIONE INTO HUMAN CELLS USING GOLD NANOPARTICLES

(76) Inventor: Calin Viorel Pop, Brooksville, FL (US)

(21) Appl. No.: 12/616,820

(22) Filed: Nov. 12, 2009

(51) Int. Cl. A61K 9/00; A61K 9/14; A61K 31/195; C12N 13/00; A01H 5/00; A61K 9/127; A61P 43/00

(52) U.S. Cl. 424/422; 424/489; 514/562; 435/173.4; 800/298; 424/450; 977/773

(57) ABSTRACT

A method of using specially designed a nanoparticles to contact and then cross the cell, nuclear and/or mitochondrial membrane of the target cell by generating a multitude of complex nanoparticle structures that resonate or vibrate at a specific frequency. Glutathione and/or other molecules or drugs are attached as molecular layers to the nanoparticle structures and the complex particle structures are delivered to the targeted cells. The glutathione and other molecules or drugs are then released from the nanoparticle structures in the destination target cell by using external radiation.

### BACKGROUND OF THE INVENTION

1. Field Of The Invention

This invention relates to methods of delivery of glutathione and other compounds inside living cells using nanogold particles as carriers. In other aspects it relates to the manufacturing of the correct nanogold particles for the above carrier purpose and, to be able to be used by itself, it relates to attaching the glutathione or other compounds to gold particles. It also relates to the delivery of glutathione—gold structure to the target living cells. In yet another aspect, it relates to the desorbtion/release of glutathione/compound from the nanogold carrier at the target site by means of resonant external methods (e.g. laser).

2. Description Of Related Art

For many years gold nanoparticles have been used as vectors for drug delivery. It is well documented by previous research that specific nanoparticles can enter various cells and the mitochondria.

There are many issues (e.g. safety, stability, delivery, penetration) that need to be considered when designing a drug delivery system based on nanoparticles. The nanocarrier particle has to be specifically designed for each carried compound in order to assure desired load, stability, penetration, safety, delivery, and elimination.

Since most of the previous research was geared towards cancer treatment, most of the products delivered are chemotherapeutic patented medications. There is little research that describes the nanocarrier delivery of nutritional supplements, non-drugs, nonpatented chemotherapeutic compounds, or patented chemotherapeutic agents using glutathione in the same time for various benefits.

We believe that glutathione, even though it is widely available and is not a chemotherapeutic agent or a patented product, is an intracellular compound of crucial significance. Optimizing glutathione delivery to the cell and mitochondria can be of tremendous importance in treating a multitude of conditions common to the aging process and chronic diseases that affect humanity.

Our invention consists in using gold nanoparticles specially designed to fix glutathione as an effective carrier for intracellular delivery of glutathione. Glutathione is a non-drug tripeptide compound that is widely regarded as the main cellular antioxidant and detoxifier. When administered, the special glutathione-nanoparticles complex can be transported to the intracellular space via the circulatory system. Once inside the target cells, the glutathione is desorbed and unloaded into the cell and the gold nanoparticles are eliminated mainly via the reticulo-endothelial system.

The release of the glutathione is realized based on a known property of gold nanoparticles, namely the plasmon band resonance. Knowing that gold particles resonate at a specific frequency we intend to use special penetrating lasers tuned to this specific frequency to help deliver and unload the carried glutathione at the target site. In addition to their action as a vector, the nanocarrier can also have therapeutic effects.

Using the same concept, elements other than glutathione (ex. drugs, chemotherapeutic agents) can be also used with or without glutathione in order to be effectively delivered to target living cells.

The manufacturing of complex gold nanoparticles has been previously described, attempted and accomplished. The novelty in our invention is the intent to prepare a specific type of gold/glutathione particles which can be carried inside living cells in a safe, reliable, and effective manner. Another novelty of our approach in the field of nanocarriers is the way the compound to be delivered is released from the carrier particles. In our case this is accomplished by subjecting the complex particle to electromagnetic radiation of a specific frequency (ex. penetrating laser).

In previous art there is also no mention of the intention or attempt to maximize, calibrate or control the desorption/unloading process in any way. We provide methods of maximizing the load of glutathione, prolonging the circulation time, choosing the best particle size and shape with maximum benefits and minimal side effects, functionalizing the particle to penetrate the cell, the mitochondria, and to target specific organs, modulating the intensity of the unloading process and the safe elimination of the gold particle through the liver and reticulo endothelial system (RES).

We also explain why this specific compound (the gold/glutathione complex) is so designed in our case that it can be delivered inside the cellular mitochondria.

While not all existing methods of generating gold nanoparticles are appropriate for our application, in general, the methods of obtaining gold/glutathione in prior art are described as being the final goal itself or a preliminary step towards more complex protein reactions or interactions: with immunoglobulins, RNA, DNA, monoclonal antibodies, gene transfer, and other complex targets or for labeling protein structures.

By contrast, we strongly believe that glutathione itself has a crucial significance in the living cell metabolism and that delivering high amounts of reduced glutathione to the cell and mitochondria can help improve the cellular function and help improve the capacity to produce energy, delay aging, reduce side effects and toxicity of medications and detoxify the organism.

The approach described in U.S. Pat. No. 6,369,206, titled “Metal Organothiol Particles”, describes in general terms the concept of such a metal organothiol particle. The gold particles in this patent are not designed or optimized to be able to have the size, degree of dispersion, and dispersion stability needed to penetrate target cells. It is also not intended to carry a high number of molecules (up to 5000 molecules of glutathione each in our case). The complex particle (glutathione/gold) is not suitable to be delivered to the target site and also intentional and controlled desorption of glutathione for therapeutic or preventive purpose is not intended.

U.S. patent application Ser. No. 11/715,563 titled “Monolayer-protected gold clusters: improved synthesis and bioconjugation” Filing date: Mar 8, 2007 also describes a method of obtaining glutathione-gold and “gold labeled rigidly held proteins”. The claims in this particular patent further limits the scope of the nanoparticles to particles with less than 10 nm in size. All the comments from the above patent (U.S. Pat. No. 6,369,206) are still applying to this particular application Ser. No. 11/715,563.

U.S. patent application Ser. No. 10/192,393 titled “Nanoparticle delivery vehicle ” describes a delivery particle designed for the purpose of the thiol group being displaced by another group for the purpose of gene expression, protein expression and nucleic acid manipulation..

The patents, journals and scholar articles so far describe prior art that uses gold nanocarriers for passive delivery of mostly proteins, genes and chemotherapeutic agents. The vast majority of applications are described in oncology and gene manipulation. Because glutathione delivery into cells was not considered, a vast field of therapeutic applications and also of prevention, anti aging, detoxification and metabolism optimization was left out because of this obsessive focus on genetic and oncology applications. This is where our invention fills a gap.

Also, the therapeutic effect of the specific gold nanoparticles themselves (without a carried molecule) when exposed to a resonant frequency has not been previously described or, to our knowledge, seriously considered. The delivery of reduced glutathione specifically, but of other agents too, using gold nanoparticles that unload when vibrating at a specific frequency has not been described so far to our knowledge and is unique to our invention. The optimization of the materials involved and the delivery process are also unique.

### SUMMARY OF THE INVENTION

The present patent application describes a method for the transport and controlled release of glutathione molecules or other therapeutic molecules into the human cells using dispersed gold nanoparticles as vectors. The proposed targeted delivery system involves three key elements: a) the innate property of glutathione molecule or other molecules to strongly adsorb on the surface of gold, b) the ability of highly dispersed gold nanoparticles with an optimum size, degree of dispersion, and surface treatment to access and enter into most cells in the body mostly via the circulatory system, and c) the release of the glutathione/therapeutic molecules in the cells as a result of the targeted excitation of the gold nanoparticles with properly tuned electromagnetic radiation.

Nano gold particles themselves vibrating at plasmon band frequency can also be effectively used as a therapeutic agent itself without carrying another substance attached on their surface.

The invention has utility in a broad range of medical applications as describes different kinds of therapeutic agents and methods. Previous uses of the nanogold carriers were in general limited only to proteins, genes and patented chemotherapeutic agents.

One advantage of our method is that it may work without a carried therapeutic substance. Other advantages derive from the fact that the delivery of glutathione may work well also for prevention, detoxification and anti aging as well as therapy, is less costly than other therapies (chemotherapy, gene manipulation, etc), more simple, less toxic and more cost effective. The delivery of glutathione or therapeutic agents is highly targeted, timed and controlled in our invention which is also unique.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings.

FIGS. 1A and 1B shows the shape of a glutathione molecule with an SH moiety in the middle. It is through this SH moiety that glutathione bonds to the carrier gold nanoparticle. The glutathione molecule is shaped in the form of an elongated T with the leg of the T very small and represented by the SH group and the rest of the molecule represented by the long T bar on top;

FIG. 2 represents gold nanoparticles in different stages of being loaded with glutathione. Our calculations show that we can attach up to 5000 glutathione molecule per nanogold particle, however this number may be increased by increasing the specific surface of the nanoparticles;

FIG. 3 shows a loaded glutathione particle entering a cell and entering the outer mithochondrial membrane;

FIG. 4 depicts the release of glutathione molecules as a result of the nanocarrier vibrating at a resonant frequency;

FIG. 5 is a graph of the specific surface area (SSA) as a function of the diameter of spherical gold particles. This graph shows that by decreasing the particle size, a progressively larger specific surface of the gold particle is obtained. For example, by decreasing the particle diameter ten times (from 30 nm to 3.0 nm) the specific surface area increases ten times (from 10.33 to 103.3 m²/g).

FIG. 6 is a graph that shows the surface plasmon band resonance of gold nanoparticles; and

FIG. 7 shows the shape of the absorbtion curve for gold nano-platelets depicting two plasmon band resonance peaks instead of just one for the case of isometric/spherical nanoparticles, where the two peaks corresponding to the length and width of the gold nanoplatelets.

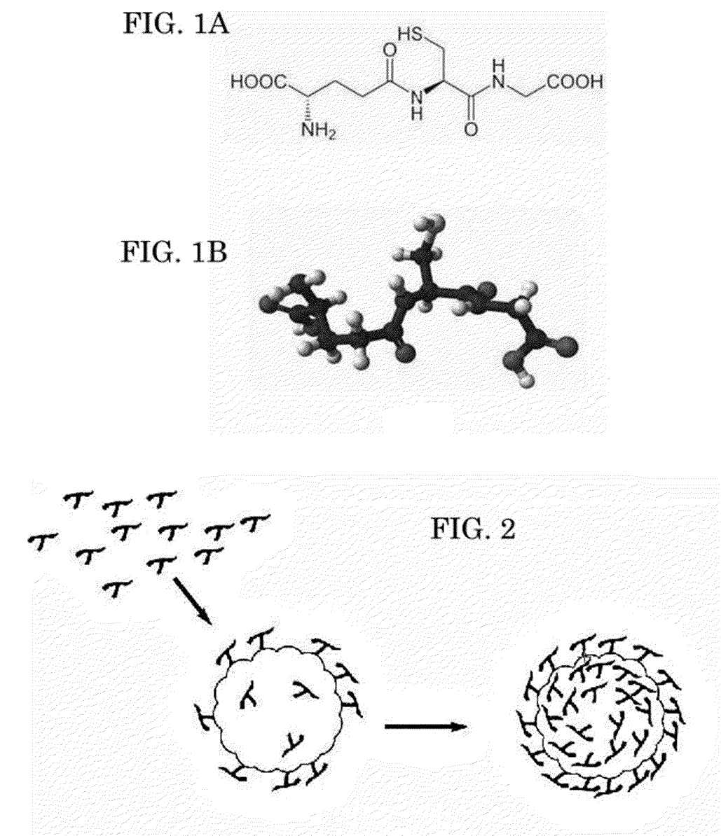

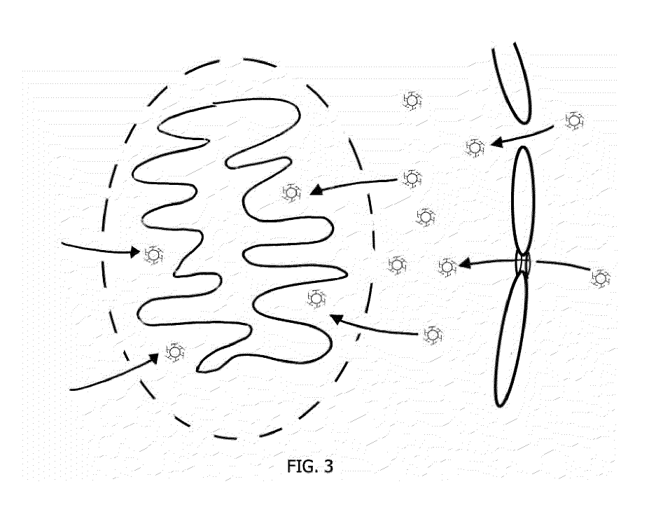

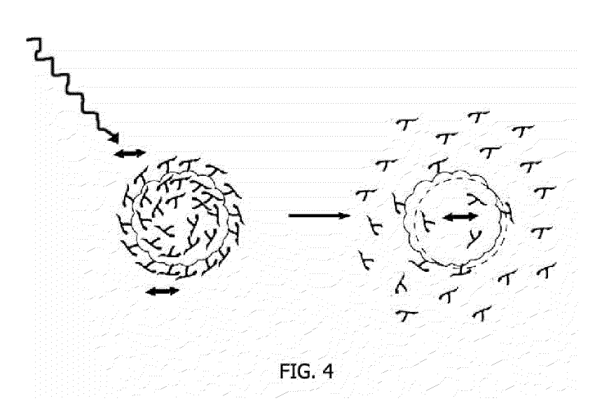

### DETAILED DESCRIPTION OF THE INVENTION

A detailed description of the present invention is better understood when it is broken down into the following several components: the importance of glutathione; the innate property of glutathione molecule to strongly adsorb on the surface of gold nanoparticles; the ability of highly dispersed gold nanoparticles with an optimum size, degree of dispersion, and surface treatment to access and enter into most cells in the body via the circulatory system; and, the release or desorbtion of the glutathione molecules in the cells as a result of the targeted excitation of the gold nanoparticles with properly tuned electromagnetic radiation. Glutathione delivered to the cytosol then enters the mithochondria (1) where most of the beneficial effects of this compound take place.

In the following detailed description of the invention we will demonstrate the following:

1. That it is highly beneficial, if not crucial for cells and living tissues to have available an abundance of reduced glutathione for essential cellular functions.

2. That we can design and produce gold nanoparticles as carriers for glutathione molecules. The gold nanocarrier will resonate at specific values of electromagnetic frequency, is safe, efficient, and will reach the target cells and enter the cells and mitochondria.

3. That we can safely desorb/unload reduced glutathione from the gold nanocarrier inside the target cells. The gold nanocarrier can later be therapeutically used by itself or can be safely eliminated from the tissues.

Methods for optimizing and controlling the above processes is provided.

### Glutathione

The glutathione molecule plays an essential role in cell metabolism (2). As a result, in both normal and abnormal physiological conditions, it may be very beneficial to increase the concentration of this important compound into the cells. The normal biochemical pathways in the human body limit how much glutathione can reach/be generated in the cells. For this reason, any method which can artificially elevate its concentration can have a major positive impact on the health and the recovery abilities of the organism.

Below is a compilation of most quoted glutathione benefits:

What is Glutathione (GSH)?

Glutathione, or GSH, is a naturally occurring protein that protects every cell, tissue, and organ from toxic free radicals and disease. It is a tripeptide of three amino acids—glycine, glutamate (glutamic acid), and cysteine. These precursors are necessary for the manufacture of glutathione within the cells.

Glutathione is a substance, the levels of which in our cells are predictive of how long we will live. There are very few other factors which are as predictive of our life expectancy as is our level of cellular glutathione. Glutathione has been called the “master antioxidant”, and regulates the actions of lesser antioxidants such as vitamin C, and vitamin E within the body. “We literally cannot survive without this antioxidant,” Earl Mindell, R.Ph., Ph.D. “What You Should Know about the Super Antioxidant Miracle”

“Without glutathione, other important antioxidants such as vitamins C and E cannot do their job adequately to protect your body against disease.” Breakthrough in Cell Defense, Allan Somersall, Ph.D., M.D., and Gustavo Bounous, M.D. FRCS(C)

“No other antioxidant is as important to overall health as glutathione. It is the regulator and regenerator of immune cells and the most valuable detoxifying agent in the human body. Low levels are associated with hepatic dysfunction, immune dysfunction, cardiac disease, premature aging, and death.” The Immune System Cure, Lorna R. Vanderhaeghe & Patrick J. D. Bouic, Ph.D.

Glutathione (L-gammaglutamyl-L-cysteinylglycine) is a tri-peptide of the amino acids cysteine, glycine, and glutamic acid. Glutathione is important in cellular respiration. A deficiency of glutathione can cause hemolysis (destruction of red blood cells, leading to anemia) and oxidative stress. Glutathione is essential in intermediary metabolism as a donor of sulfhydryl groups which are essential for the detoxification of acetaminophen. [PDR Medical Dictionary. Spraycar. 1999]

Glutathione is the major endogenous antioxidant produced by the cell. Glutathione participates directly in the neutralization of free radicals, reactive oxygen compounds, and maintains exogenous antioxidants such as vitamins C and E in their reduced (active) forms. In addition, through direct conjugation, glutathione plays a role in the detoxification of many xenobiotics (foreign compounds) both organic and inorganic. Glutathione is an essential component of the human immune response.

As an antioxidant, glutathione is essential for allowing lymphocytes to express their full potential, without being hampered by oxyradical accumulation during the oxygen requiring development of the immune response. In a similar fashion, GSH delays the muscular fatigue induced by oxyradicals during the aerobic phase of strenuous muscular contraction. As a detoxification agent, glutathione has been demonstrated to be effective against a number of xenobiotics, including chemical pollutants, various carcinogens and ultraviolet radiation.

“A review article published in the Annals of Pharmacology stated that glutathione is important in DNA synthesis and repair, protein and prostaglandin synthesis, amino acid transport, detoxification of toxins and carcinogens, enhancement of the immune system, and protection from oxidation and enzyme activations.” The Immune System Cure, Lorna R. Vanderhaeghe & Patrick J. D. Bouic, Ph.D.

Research suggests that abnormally low glutathione levels may increase the risk for a Heart attack. Eric Topol, MD, NEJM

“Glutathione has potent anti-viral properties—if tissue and serum glutathione levels are significantly increased, the replication of most pathogens are slowed or stopped. Conversely, glutathione deficiency produces a pro-viral effect.” Paul Cheney, M.D., Ph.D. and expert in the treatment of Chronic Fatigue Syndrome. Transcribed from a workshop presentation on the clinical management of Chronic fatigue Syndrome. Lymphocytes, cells vital for effective immune function, depend on GSH for their proper function and replication.

### IMMUNOLOGY 61: 503-508 1987

As we age, there is a precipitous drop in GSH levels. Lower glutathione levels have been implicated in many diseases associated with aging.

### Journal of Clinical Epidemiology 47: 1021-28 1994

Antioxidants are well documented to play vital roles in health maintenance and disease prevention. GSH is your cells' own major antioxidant.

### Biochemical Pharmacology 47: 2113-2123 1994

GSH plays a role in eliminating many carcinogens as well as maintaining immune function.

### Cancer Letters 57: 91-94 1991

Strong muscular activity, such as that experienced by athletes, generates oxyradicals [free radicals] leading to muscle fatigue and poorer performance. Glutathione (GSH) neutralizes these radicals.

### Sport Medicine 21: 213-238, 1996

GSH detoxifies many pollutants, carcinogens, and poisons, including many in fuel exhaust and cigarette smoke. It retards damage from radiation such as seen with loss of the ozone.

### Annual Reviews of Biochemistry 52: 711-780 1983

Most of the cellular glutathione (GSH) (85-90%) is present in the cellular matrix (cytosol) from where it is transported to the mithochondria. With the exception of bile acid, extracellular concentrations of GSH are relatively low.

As generators of free radicals, mitochondria have antioxidant defense systems to counteract oxidative stress. Because it lacks self sufficient antioxidant mechanisms, mitochondria depend on glutathione for this function. GSH is an endogenous combatant against H2O2 (Fernandez-Checa et al., 1998). Mitochondria lacks the enzymes needed to synthesize GSH, so GSH must be transported into the mitochondria.

Increasing GSH levels in the mitochondria is an outmost important therapeutic approach to preventing cell death in oxidative stress-linked, age-dependent neurodegenerative disorders (1).

Glutathione, an essential cellular antioxidant required for mitochondrial function, is not synthesized by mitochondria but is imported from the cytosol.

Increase of extra-mitochondrial glutathione promotes uptake and rapid exchange that occurs between mitochondrial and cytosolic glutathione. (ii) lowering of cytosolic glutathione levels (produced by administration of buthionine sulfoximine) decreases export of glutathione from mitochondria to cytosol to preserve reserves of glutathione, and (iii) administration of glutathione esters increases glutathione levels in mitochondria more than those in the cytosol (1).

Due to the above comment explaining the rapid exchange between the cytosolic and mitochondrial GSH, the penetration of the glutathione gold complex particles inside the outer mithochondrial membrane may not even be necessary.

Unlike most other membranes, the outer mitochondrial membrane seems to be freely permeable to various small molecules and has therefore been called ‘leaky’. The intactness of this membrane is demonstrated by observations which show it to be impermeable to large polymers. This ‘sieving’ property is consistent with the presence of size-selecting units such as channels. The channels are big enough so that the gold, glutathione complex can pass through the outer membrane.

Cytochrome c is a highly conserved protein across the spectrum of species, found in plants, animals, and many unicellular organisms. This, along with its small size (molecular weight about 12,000 daltons), makes it useful in studies of cladistics. Its primary structure consists of a chain of about 100 amino acids—and crosses the inner membrane easily. It is of clear consequence that in absence of other factors a much smaller molecule like glutathione made out of only three aminoacids, having only 307 daltons and three small aminoacids should penetrate the inner membrane easily.

A literature search provided that GSH was entering cells of kidneys, lungs/alveolar, gastric and intestinal cells. It was found that renal epithelial cells and intestinal epithelial cells contain a sodium dependent uptake system for intact GSH (8).

Until the late 1970s and perpetuated long after that time, the dogma was that GSH is not transported into cells as an intact tripeptide. A thorough review of the literature (3, 4, 5, 7) reveals that GSH is indeed transported into cells intact.

Only one observation of seven patients (6) was used to document that glutathione can not be absorbed intact from intestines, fact that contradicts clinical findings and scientific studies other than the one above mentioned.

Despite current beliefs that only glutathione precursors elevate intracellular glutathione, a review of current studies reveals that Glutathione itself was several times more efficient in improving the antioxidant and detoxification function compared to taking the three component amino acids separately (precursors). (9). GSH was three times more efficient than the precursors in raising the intracellular glutathione concentration.

After reviewing the literature on the subject of intestinal aminoacid and protein absorbtion we find in a hyperlink physiology textbook from Colorado state university making the statement that: “There is virtually no absorption of peptides longer than four amino acids. However, there is abundant absorption of di- and tripeptides in the small intestine”. Considering that GSH is a very small tripeptide of only 307 daltons that is built from three of the smallest aminoacids in existence it can be logically deducted that GSH is indeed absorbed by the intestinal lining.

Further review of literature reveals that gold nanoparticles are absorbed by the intestinal lining. Studies were performed up to 198 nm gold particles size for skin and intestinal lining absorbtion in rats (32,41) . As expected the bigger the nanoparticle size the lesser the gastrointestinal or transdermal uptake was.

Our conclusion is that it is highly beneficial, if not crucial, for cells and living tissues to have available an abundance of reduced glutathione for essential functions.

### Nano Gold Particles

Nanoparticles generally refer to particles with diameters or size ranging usually from 1 to 100 nm, rarely to a few hundred nm.

Because of chemical inertness gold has been used internally in humans for the past half a century. There is ample evidence that gold nanoparticles are very effective delivery vectors for many drugs (10,11,12,13,) They are very well tolerated by the body, can effectively access all organs, and can also enter the intracellular space. In order to be able to effectively travel through the body the gold nanoparticles must not be too large (typically less than ˜35 nm) and remain highly dispersed in the blood and other body fluids. Also, it is critically important that the particles are well tolerated by the body. This can be achieved, for example, by covering part of the surface of gold with derivatized PEG molecules (14) Several factors have been recognized to govern the cellular uptake efficiency of nanoparticles, such as size (15,16,) shape (15) and surface properties (17,18,19,20,).

Because the cell membrane is negatively charged, positively charged nanoparticles are generally found to have higher uptake efficiencies. (18,20)

Gold nanoparticles are excellent delivery vectors and carriers to cancer cells as they selectively accumulate up to 5-600 times more in cancer cells than in noncancerous cells. Furthermore, it has been shown that in certain cancer lines the gold nanoparticles, even in the absence of any specific functionalization, have been able to induce apoptosis (36).

### Safety Of Nanogold Particles

As opposed to cytotoxic effects of other nanomaterials like quantum dots and carbon nanotubes, the use of gold nanoparticles as a carrier and delivery agent stems from the absence of reports on gold nanoparticles induced toxicity (36,37). Some studies show that only cationic particles are moderately toxic at core size around 2 nm, whereas anionic particles are quite nontoxic (40) including for the blood brain barrier cells. However, such very small sized gold nanoparticles were found to be non toxic when administered to mice for tumor therapy (Hainfeld et al 2004). Though there are studies showing that the carried agent is sometimes cytotoxic, however, gold particles by themselves are non toxic (38). There are also some studies showing that a particular size—of 1.4 nm —that for steric reasons may present an in vitro concern of a mild growth inhibition, however sizes very close to 1.4 nm, smaller or larger clusters (1.2 nm or 1.8 nm are many times safer) do not have this problem (39). Smaller gold particles (Tauredon) or larger up to 15 nm gold colloids were comparatively nontoxic, irrespective of the cell type tested. There are many gold compounds currently commercially available that have low toxicity.

Nanoparticles, of a few nm in size, may reach inside of folding biomolecules, especially the 1.4 nm size , a situation not possible for larger particles.

With silica nanoparticles of about 42 nm, gene transfer was obtained with very low cell toxicity (Ravi Kumar et al 2004) and particles with size of about 40-50 nm—magnetic nanoparticles coated with PEG were quite well taken up by endocytosis (Gupta and Curtis 2004)

It is shown in the literature that gold nanoparticles smaller than 35 nm are usually safe (33,34) and are later safely eliminated from the through RE system. Some of the safety issues come from the attached molecules rather than the gold particles themselves.

An excellent tolerance of high concentrations of injectable gold agents, for example Myochrisine, was demonstrated in humans.

It is significant that GSH is considered one of the greatest detoxifier and toxicity prevention agent in existence, therefore the toxicity of the glutathione gold nanoparticles would be highly reduced and likely can be significantly reduced even further if the size and specific functionalization of the particle is carefully chosen. Safety issues usually arise at sizes higher than 40 nm, the very specific size of 1.4 nm and then maybe at much lower sizes. For our application a sweet spot around 10-20 nm seems to provide the greatest benefits with minimal side effects.

### Gold Nanoparticles Entering The Cells

It is an established fact that nanoparticles enter or transfect into the cells by phagocytosis or mainly by endocytosis for the size of nanoparticles that we are discussing. By endocytosis the particle is gradually embedded by deforming the membrane and later absorbed or endocytosed into the cell. The endocytosis process can be promoted by certain functionalities on the nanoparticle surface (Zhang et al., 2004). For nucleus penetration the particle can be functionalized with yet another specific peptide. There can also be a two step functionalization: the first to enter the cell easier, the second one to target and enter the nucleus or the mitochondria. Fullerenes are an example of nanostructures that preferentially bound to mitochondria.

One particular study showed that, after administration, 10 nm particles were present in various organ systems including blood, liver, spleen, kidney, testis, thymus, heart, lung and brain, whereas the larger particles were only detected in blood, liver and spleen. The results demonstrate that tissue distribution of gold nanoparticles is size-dependent with the smallest 10 nm nanoparticles showing the most widespread organ distribution (42). Gold particles are shown in studies to also penetrate the mitochondrial membranes and thus enter the mitochondria.

Once the glutathione is attached on the surface of the gold it does not have ionizable groups so it is neither anion or cation. The whole gold-glutathione complex can be subsequently charged due to ions adsorbed from the surrounding environment (Cl−, anion or Na+, H+ which are cations) thus electrostatically influencing the particle for a desired behavior.

It has been reported that inhaled nanoparticles also reach the blood and may reach other target sites such as the liver, heart or blood cells (Oberdorster G et al 2002, MacNee et al 2000, Kreyling et al 2002).

Nanoparticles may translocate through membranes. There is little evidence for an intact cellular or sub-cellular protection mechanism against nanoparticles entering the cells.

It is demonstrated that glutathione is well absorbed on nanogold particle and that the resulting nanostructure is stable.(27,28) It is also documented that nanogold can pass through the intestinal wall (8,29) and can be safely injected intravenously or intra arterial. There are presently many commercial applications of nanogold particles especially in the field of oncology, the gold being used as a nanocarrier.

When research is thoroughly considered we can clearly conclude that we can deliver glutathione to the cell cytosol, using nanogold as a carrier.

It is found that molecules of up to 1000 daltons can diffuse freely across the outer mitochondrial membrane. (30) Precursor proteins also can passively diffuse across the outer mitochondrial membrane in the absence of ATP. No direct experimental information on the size of the protein import channel in the inner membrane has been obtained thus far.

Results show that the internal diameter of the protein import channel of the outer membrane is between 20 Å and 26 Å with an average of 22 Angstroms (2.2 nm). (30)

The internal diameter of the inner membrane import channel is smaller than that in the outer membrane. It follows that nanoparticles smaller than 2 nm in absence of other detrimental factors can also passively diffuse inside the outer membrane of the mitochondria.

### Glutathione Adsorbtion On Gold Nanoparticles

Gold nanoparticles has been used for a long time as vectors to deliver compounds and drugs to different areas of the body. The majority of present uses of gold nanoparticles are patented drugs delivery.

Due to special chemical physical properties, gold nanoparticles can deliver other specific compounds, thiols in particular. Thiol group is an anchor for gold.

Because of a particular molecular structure, glutathione (G—SH) is an ideal compound to be absorbed on gold nanoparticles.

Why is this useful? Glutathione attached to gold can be introduced in living tissues in a reduced form thus highly enhancing the antioxidant, immuno-regulating, detoxification and antiaging properties of the glutathione.

### Preparation And Comments On Gold/Glutathione Particles

From the chemical point of view, the glutathione molecule has a sulfur containing functional group that interacts very strongly with gold. As a result, glutathione molecules are adsorbed readily when in contact with clean gold surfaces. For the adsorption to occur, the surface of the latter must be either free of other adsorbents or be covered with molecules which have weaker interactions with the gold surface.

For this reason, the method used in the preparation of gold nanoparticles is critical in the adsorption of the glutathione molecules on their surface. Preferably, the gold particles should be prepared in the absence of any dispersant or surfactant.

Several methodologies acceptable from this point of view are available (the citrate method reported by Frens, Goia method with ascorbic acid. (21,22,23)) although they yield very low gold concentration and each has a relatively limited range of particle sizes. Other methods use dispersing agents to alleviate these disadvantages. In these cases the gold particles can absorb glutathione molecules only if the additives used in the precipitation contain functional groups (such as carboxylic, hydroxyl, amino), which give relatively weak interactions with the gold and thus can be easily displaced by the thiol group of glutathione.

In some situations it is of interest to also attach other molecules to the monolayer such as folic acid. It was demonstrated by Zhang et al 2003, that there is a five time increase in cellular uptake of nanoparticles by breast cancer cells if folic acid was present on their surface. Folic acid segment functionalization can be used as a targeting agent for cancer cells expressing the folate receptor.

An important advantage of using gold as a vector for the delivery of glutathione relates to the ability to maintain the molecule in its reduced form (GS) which is the most biologically active. Indeed, once the molecule is adsorbed on the surface of gold, the sulfur containing functional group it is not anymore available for pairing with a second glutathione molecule to form the inactive/oxidized glutathione disulfide (GSSG).

Although it is desirable to maximize the amount of glutathione adsorbed on the surface of gold, it is also essential that a fraction of the surface of the core nanoparticles is covered with molecules that provide the stability of the colloidal dispersion in the physiological fluids and the compatibility of the vector particles with the environment in the body.

Thiol-derivatized polyethylene glycol (PEG) fragments have been successfully used by Paciotti et al (11) in the case of the delivery of antitumor drugs using ˜32 nm spherical gold nanoparticles prepared by the Frens citrate method. Similarly, a judicious ratio of co-deposited glutathione molecules and PEG-thiol must be achieved for a successful delivery using gold nanoparticles as a vector.

Particles covered with PEG can resist opsonization, effectively creating “stealth particles” with extended circulation times. Most gold particles can be found hours and days after administration in the liver, spleen and lungs at least partially as a contribution of the above organs to the RES system that clears the particles out of the body.

Functionalization and coating of the gold nanoparticles can increase or decrease the clearance or uptake by the RES. For example the PEG coating will also improve the non specific cellular uptake, improving the circulation time and helping delivery to sites other than liver spleen and lungs. The ideal functionalization would be two steps: first with PEG to improve nonspecific cellular uptake and the with a second agent to speed up the clearance of the gold nanoparticles from the body. Most clearance is performed through the liver and bile and some through other means including kidneys. In cases where the above mentioned organ targeting is needed, the above features could be helpful in accomplishing these results.

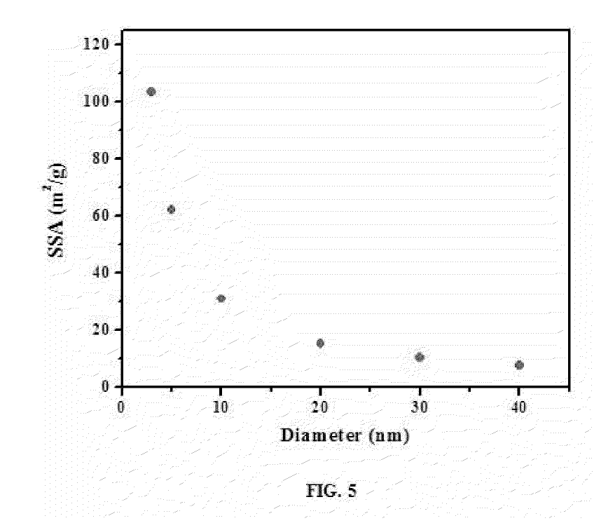

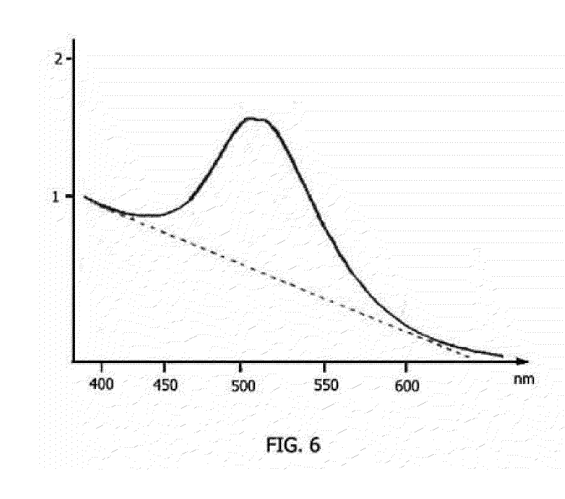

While the maximum loading of glutathione (ng/mg Au) depends on the ratio between the surface occupied by glutathione and the co-additive surfactant (PEG), equally important are the size and the shape of the gold nanoparticles. It is widely agreed (11,12,24) that, in order for Au particles to be suitable for ‘in vitro’ delivery systems, their maximum dimension can not exceed ˜35nm (assuming that particle aggregation is completely prevented). By decreasing the particle size, a progressively larger specific surface of the gold (see FIG. 5) is obtained. Indeed, as it can be seen in the graph in FIG. 5, by decreasing the particle diameter ten times (from 30 nm to 3.0 nm) the specific surface area increases ten times (from 10.33 to 103.3 m²/g).

Despite their increased specific surface area and capability of adsorbing larger amounts of glutathione, very small particles are not always suitable as delivery vectors mainly because they easily aggregate in the very complex fluid matrices in the body due to their very high surface energy.

An alternative tool to increase the specific surface area of gold and the amount of adsorbed glutathione without an increased aggregation risk is to use gold anisotropic particles (platelets, rods). Indeed, the specific surface for a 30 nm spherical particle is ˜10.4 m²/g while for a hexagonal platelet with the same size (30 nm) and a thickness of 4 nm the value is ˜33.9 m²/g, a 330% increase. Gold nano-platelets have two plasmon band resonance peaks instead of just one, the two peaks corresponding to the length and width of the gold nanoplatelets as depicted in FIG. 7.

The loading of glutathione on the gold vector can be easily estimated. Assuming that the surface occupied (projection) by a molecule is ˜0.5 nm² the loading of glutathione is ˜1,020 μg for each square meter of gold. If the fraction of the surface covered with glutathione represents only 80% of the total surface of gold (the rest being reserved for the PEG-thiol), 1.0 mg of 30 nm Au particles adsorb 8.5 μg of glutathione (1020×0.8×10.4), while 1.0 mg of 3 nm Au particles absorb ˜85 μg (1020×0.8×104).

In conclusion, we can design and produce a safe gold nanoparticle carrier for glutathione. This gold nanocarrier will resonate with an electromagnetic frequency, it would be safe, efficient and would reach the target cells and enter the cells and mitochondria.

### Desorbtion/Unloading Of Glutathione

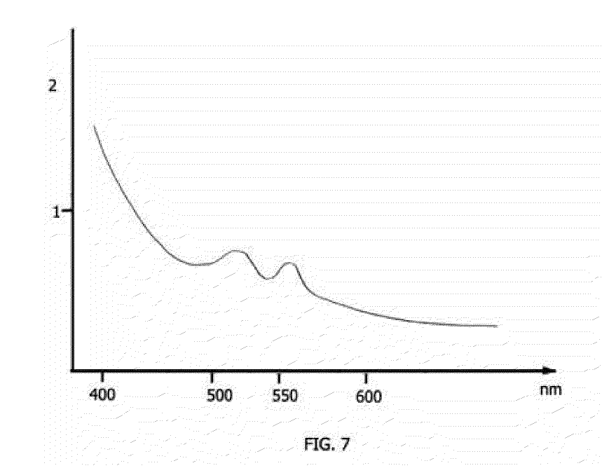

Gold and silver nanoparticles have a unique ability to share electrons on the surface of the nanoparticle. This gives rise to specific absorbtion of electromagnetic radiation that looks like a peak on a graph (FIG. 6). The top of the peak is the frequency of maximum resonance, however there is a resonant band or interval in which the nanoparticles can resonate. The frequency does not have to be extremely exact for this effect to occur. This effect of absorbing and resonating to a specific frequency and to a certain frequencies that are close to the maximum frequency is well known in the industry and is called surface plasmon band resonance of gold and silver nanoparticles.(25,26)

This phenomenon is widely used in the industry to verify that indeed there are nanoparticles in solution and to express how narrow the size distribution of the nanoparticles is. The higher and the narrower the peak on the graph, the more defined size nanoparticle distribution in a narrow range exists in a solution.

For example spherical nanogold particles absorb electromagnetic radiation with a wavelength having a peak at 510 nm (at 3 nm particle size) and 522 nm (at 35nm particle size). Thus, a penetrating laser or even visible light with an emission band in these ranges should be used in order to produce resonance of the gold or silver nanoparticles.

By subsequently exposing the target area to external vibrations that match the resonant band of the specially designed carrier nanoparticles, the gold nanoparticles shall start vibrating and emitting heat or other types of radiations.

The amount of heat and vibration triggered into nanoparticles can be adjusted by modulating the resonant frequency and the intensity/amplitude of the external frequency band.

Once nanoparticles start vibrating with a higher and higher intensity, glutathione (or any other carried element) will start to fall off and be unloaded from the nanoparticle surface once a specific intensity/amplitude of vibration is achieved. The amount and completeness of unloading depends on many factors such as the size of the particle, the intensity of the external radiation and its penetration, the type of ligand(s), the type of tissue, etc. Depending of the above factors (and others) a reliable exposure protocol (frequency, intensity, modulation, site, etc) can be established for different tissues for the specific purpose of unloading a specific amount of glutathione to the targeted tissue. Nanoparticles have been used in mass spectrometric analyses primarily to facilitate the laser desorption/ionization of compounds of interest.

The method of using radiation to activate drugs is not new. It is utilized and can be found under different names like PDT (Photo Dynamic Therapy), nanoPDT, bio-photonix etc. (31,32)

In oncology, photodynamic therapy (PDT) combines a drug (called a photosensitizer or photosensitizing agent) with a specific type of light to kill cancer cells. PDT essentially has three steps. First, a light-sensitizing liquid, cream, or intravenous drug (photosensitizer) is applied or administered. Second, there is an incubation period of minutes to days. Finally, the target tissue is then exposed to a specific wavelength of light that then activates the photosensitizing medication.

In our case the PDT is somehow modified in the sense that it is not the photo sensitizing medication that is activated but instead is the carrier nanoparticle. However, sometimes the nanoparticles themselves can act as therapeutic agent.

PDT is currently used in a number of medical fields including oncology (cancer), dermatology (skin), and cosmetic surgery.

The new element we propose is the activation, excitation and entrainment of the nanoparticles at their specific plasmon band resonance frequency band in order for nanoparticles to vibrate with increased amplitude and, if loaded with a carried substance, to unload this substance at least partially.

Besides nanogold being used as a carrier of other materials and proteins is very important to remember that gold accumulates intensely in areas with tumors injury or inflammation. Since gold alone in sizes and clusters much higher than 100 nm is already effectively used for treatment of inflammatory conditions like Rheumatoid arthritis we conclude that nanogold particles could be effectively used at specific nanoparticle sizes to obtain a therapeutic effect. The vibration at plasmon band resonance would amplify this therapeutic effect even further. Due to this phenomenon, nanogold vibrating at plasmon band frequency can be effectively used as a therapeutic agent itself without carrying another substance attached on its surface.

The therapeutic effect of effect of gold nanoparticles themselves (without a carried molecule) vibrating at a resonant frequency has not been described so far to our knowledge and is unique to our invention

It is important to mention that gold nano platelets offer additional advantages since they have two absorption bands and peaks (corresponding to the transversal and longitudinal oscillations) and have therefore double excitation band (FIG. 7). In theory they can resonate by vibrating in one direction to deliver/desorb one compound attached to its surface and then vibrating in another direction usually 90 degrees apart to desorb the second compound.

### Gold Elimination

There are indications by several preclinical models that suggest that the electrostatically stabilized particles are taken up by hepatocytes (Hardonk et al. 1985; Renaud et al. 1989), not Kupffer cells, excreted into the bile and expelled from the body in feces (Hardonk et al. 1985; Renaud et al. 1989). The gold glutathione nanoparticle is an electrostatically stabilized particle, thus speeding up elimination through the liver and bile. Two key factors influence the clearance of gold particles. First, smaller colloidal gold particles stabilized with either a protein—as an example glutathione is a small molecule—very small—or a polymer were preferentially taken up by the hepatocytes and ultimately excreted into the bile and eliminated in the feces (Hardonk et al. 1985; Renaud et al. 1989).This is reason to believe that the coating with thousands of glutathione molecules will speed up the elimination of these particles from the organism. Also, blocking Kupffer cell activity with gadolinium chloride also increased the fraction of particles cleared by the hepatocytes (Renaud et al. 1989). (11). In other words the gold can be eliminated in various proportions through the liver and bile and also through the RES.

The gold itself can be designed to remain in the organism for a period of time that is longer or shorter depending mostly of the size of the particles and other factors as well. As mentioned above, functionalization and coating of the gold nanoparticles can increase or decrease the clearance or uptake by the RES.

For example, the PEG coating will also improve the non specific cellular uptake, improving the circulation time and helping delivery to sites other than liver spleen and lungs. As mentioned above, the ideal functionalization of a gold-glutathione particle would be in two steps: first with PEG to improve nonspecific cellular uptake and then with a second agent to speed up the clearance of the gold nanoparticles from the body. Most clearance is performed through the liver and bile and some through other means including kidneys.

The gold particles themselves, with or without glutathione can further play a role in the treatment of some specific illness (rheumatoid arthritis is a well documented condition). The role of activated gold—meaning gold particles vibrating to it's plasmon band resonance in the in vivo tissues is itself a wide unexplored open field in the therapy of many medical conditions.

The end result of gold carried glutathione to the cells is that the cell cytosol is thus receiving a significant amount of reduced glutathione—up to 5000 or more molecules for each carrier gold nanoparticle. Reduced glutathione is then transported into the mitochondria according to the literature (1,35) where it plays an essential role in vital processes: antioxidant, immunity, antiaging, energy, etc.

In conclusion we can safely desorb/unload reduced glutathione from the gold nanocarrier inside the target cells. The gold nanocarrier can be used by itself or can be safely eliminated from the tissues. We also provided methods for optimizing and controlling the above processes.

From an abundance of scientific articles, and a thorough research it is now clear to us that a surge of reduced glutathione in the cell mitochondria can have tremendous beneficial health effects on any organism. Our method accomplishes this result.

While there have been shown and described and pointed out the fundamental novel features of the invention as applied to the preferred embodiments, it will be understood that various omissions and substitutions and changes of the form and details of the apparatus illustrated and in the operation may be done by those skilled in the art, without departing from the spirit of the invention.

### PUBLICATIONS REFERENCED ABOVE:

1. Proceedings of the National Academy of Sciences of the USA

### PNAS, Volume 87, Issue 18, pp. 7185-7189

2. Recent advances in nutritional sciences: glutathione metabolism and its implications for health

### Guoyao Wu, Yun-Zhong Fang, Sheng Yang, Joanne R. Lupton and Nancy D. Turner

### J. Nutr. 134:489-492, March 2004

3. Glutathione-nutritional and pharmacologic viewpoints
   A comprehensive review of GSH in foods and its gastrointestinal absorption.

### Valencia E, Marin A, Hardy G.

### Part IV. Nutrition 2001; 17: 783-784.

4. Fate of dietary glutathione: disposition in the gastrointestinal tract.

### Hagen T M, Wierzbicka T G, Sillau A H, et al.

### Am J Physiol 1990; 259: G524-G529.

5. Effect of orally administered glutathione on glutathione levels in some organs of rats: role of specific transporters.

### Favilli F, Marraccini P, Lantomasis T, Vincenzini M T.

### Br J Nutr 1997; 78: 293-300.

6. The systemic availability of oral glutathione.

### Witschi A, Reddy S, Stofer B, Lauterburg B H.

### Eur J Clin Pharmacol 1992; 43 (6): 667-669.

7. Absorption of glutathione from the gastrointestinal tract.

### Hunjan M K, Evered D F.

### Biochim Biophys Acta 1985; 815: 184-188.

8. Protection against paraquat induced injury by exogeneous GSH in pulmonary alveolar type II cells

### Tory Hagen, Lou Brown, Dean Jones, Emory School of Medicine

### Biochemical Pharmacology, 1986, 35:24, pp 4537-4542

9. Glutathione uptake and protection against oxidative injury in isolated kidney cells

### Tory M. Hagen, Tak Yee Aw, Dean P. Jones

### Kidney International, Vol. 34 (1988), pp. 74-81

10. Colloidal gold: A novel nanoparticle vector for tumor directed drug delivery

### Giulio F. Paciotti, Lonnie Myer, David Weinreich, Dan Goia, Nicolae Pavel, Richard E. McLaughlin, Lawrence Tamarkin

### Drug Delivery 2004 11:3, 169-183

11. Colloidal gold nanoparticles: a novel nanoparticle platform for developing multifunctional tumor-targeted drug delivery vectors

### Giulio F. Paciotti , David G. I. Kingston , Lawrence Tamarkin

### Nanobiotechnology , 2006, 67:1, p 47-54

12. Gold and nano-gold in medicine: overview, toxicology and Perspectives

### N R Panyala, E M Pena-Mendez, J Havel

### J Appl Biomed, 2009, 7: p 75-91

13. Gold nanoparticles: preparation, functionalisation and applications in biochemistry and immunochemistry

### Lev A Dykman and Vladimir A Bogatyrev

Russ. Chem. Rev. 2007, 76:2 181-194
14. Attachment of functionalized poly(ethylene glycol) films to gold surfaces
   Hongbo B. Lu, Charles T. Campbell, and, David G. Castner

### Langmuir 2000 16 (4), 1711-1718

15. Measuring the flexibility of immunoglobulin by gold nanoparticles

### Chithrani B D, Ghazani A A, Chan W C W.

### Nano Lett. 2006; 6:662-668

16. Nanoparticle mediated cellular response is size dependent

### Jiang W, Kim B Y S, Rutka J T, Chan W C W. Nat.

### Nanotechnol. 2008; 3:145-150

17. Transferrin mediated gold nanoparticle cellular uptake

### Yang P H, Sun X, Chiu J F, Sun H, He Q Y.

### Bioconjugate Chem. 2005,16:3 , p 494-496.

18. Nanoparticle interaction with biological membranes:Does nanaotechnology present a Janus face?

### Leroueil P, Hong S, Mecke A, Baker J, Orr B, Banaszak Holl M.

Acc. Chem. Res. 2007, 40:5, p 335-342.
19. Gold nanoparticle mediated transfection of mammalian cells

### Sandhu K K, McIntosh C M, Simard J M, Smith S W, Rotello V M.

### Bioconjugate Chem. 2002, 13:1 p 3-6

20. Inorganic particles as carriers for efficient cellular delivery

### Xu Z P, Zeng Q H, Lu G Q, Yu A B.

Chem. Eng. Sci. 2006, 61, p1027-1040
21. Size controlled synthesis of gold nanoparticles using photochemically prepared seed particles

### Sau T. K; Pal A; Jana N. R.; Wang Z. L.; Pal T

### Journal of Nanoparticle Research 2001, 3:4, p 257-261(5)

22. Preparation of gold colloid using pyrrole-2-carboxylic acid and characterization of its particle growth

### Takeshi Sakura, Yukio Nagasaki

### Col.Polymer Science 2007, 285:12, p 1407-1410

23. Turkevich method for gold nanoparticle synthesis revisited

### J. Kimling, M. Maier, B. Okenve, V, Kotaidis, H. Ballot, and A. Plech

J. Phys. Chem. B, 2006, 110:32, pp 15700-15707
24. Therapeutic possibilities of plasmonically heated gold nanoparticles

### Dakrong Pissuwan, Stella Valenzuella, Michael Cortie

### Trends in Biotechnology , 2006, 24:2 p 62-67

25. Surface plasmon resonance of colloidal Au-modified gold films
   L. Andrew Lyon, Michael D. Musick, Patrick C. Smith, Brian D. Reiss, David J. Peña Michael J. Natan

### Sensors and Actuators B: Chemical, 1999, 54:1-2, p 118-124

26. Plasmon-enhanced fluorescence of dye molecules

### Monica Iosin, Patrice Baldeck and Simion Astilean

Nucl Instr. & Meth Physics Research ;B: Beam Interactions with Materials and Atoms
2009, 267: 2, p 403-405
27. Giant gold-glutathione cluster compounds: intense optical activity in metal-based transitions

### T. Gregory Schaaff and Robert L. Whetten

J. Phys. Chem. B 2000, 104, 2630-2641
28. Medicinal gold compounds

### R V Parish, S M Cottrill

Gold Bull, 1987, 20, p1,2
29. Gastrointestinal persorption and tissue distribution of differently sized colloidal gold nanoparticles

### Julian F. Hillyer, Ralph M. Albrecht

### J Pharm Sciences 90:12, p 1927-1936

30. The dimensions of the protein import channels in the outer and inner mitochondrial membrane

### Michael Scwartz and Andreas Matouschek

### PNAS 1999 96:13086-13090

31. Brain cancer diagnosis and therapy with nanoplatforms

### Yong-Eun Lee Koo, G. Ramachandra Reddy, Mahaveer Bhojani, Randy Schneider, Martin A. Philbert, Alnawaz Rehemtulla, Brian D. Ross and Raoul Kopelman

### Adv Drug Delivery Rev 2006, 58:14, p 1556-1577

32. Vascular targeted nanoparticles for imaging and treatment of brain tumors

### G. Ramachandra Reddy, Mahaveer S. Bhojani, Patrick McConville, Jonathan Moody

Clin. Cancer Research 2006, 12, p 6677-6686
33. Aqueous synthesis of gold nanoparticles and their cytotoxicity in human dermal fibroblasts-fetal
   Yinghua Qu and Xiaoying Lü
   Biomed. Mater. 2009, 4(2):2007. Epub 2009 Mar. 4
34. Toxicology of nanoparticles: A historical perspective
   Günter Oberdörster; Vicki Stone; Ken Donaldson

### Nanotoxicology, 2007, 1:1, p 2-25

35. Mitochondrial glutathione: importance and transport.

### Fernandez Checa, J C : Kaplowitz, N : Garcia Ruiz, C : Colell, A

### Semin-Liver-Dis. 1998; 18:4, p 389-401

36. Cell selective response to gold nanoparticles

### H. Patra, S. Banerjee, U. Chaudhuri, P. Lahiri, A. Dasgupta

### Nanomedicine, 3: 2, p 111-119

37. Gold Nanoparticles Are Taken Up by Human Cells but Do Not Cause Acute Cytotoxicity

### Connor, Ellen E | Mwamuka, Judith | Gole, Anand | Murphy, Catherine J | Wyatt, Michael D

### Small. 2005, 1: 3, p. 325-327

38. Cellular Trajectories of Peptide-Modified Gold Particle Complexes: Comparison of Nuclear Localization Signals and Peptide Transduction Domains

### Alexander G. Tkachenko, Huan Xie, Yanli Liu, Donna Coleman, Joseph Ryan, Wilhelm R. Glomm, Mathew K. Shipton, Stefan Franzen, Daniel L. Feldheim

### Bioconjugate Chem., 2004, 15 (3), pp 482-490

39. Size-Dependent Cytotoxicity of Gold Nanoparticles
   Yu Pan, Sabine Neuss, Annika Leifert, Monika Fischler, Fei Wen, Ulrich Simon, Günter Schmid, Wolfgang Brandau, Willi Jahnen-Dechent

### Small, 2007, 3:11, p 1941-1949

40. Toxicity of Gold Nanoparticles Functionalized with Cationic and Anionic Side Chains

### Catherine M. Goodman, Catherine D. McCusker, Tuna Yilmaz, and Vincent M. Rotello

### Bioconjugate Chem. 2004, 15, p. 897-900

41. In vitro permeation of gold nanoparticles through rat skin and rat intestine: effect of particle size.

### Sonavane G, Tomoda K, Sano A, Ohshima H, Terada H, Makino K.

### Colloids Surf B Biointerfaces. 2008 Aug 1;65(1):p 1-10

42. Particle size-dependent organ distribution of gold nanoparticles after intravenous administration
   Wim H. De Jong, Werner I. Hagens, Petra Krystek, Marina C. Burger, Adriënne J. A. M. Sips and Robert E. Geertsma

### Biomaterials, Volume 29, Issue 12, April 2008, Pages 1912-1919

Webpages
   http://www.vivo.colostate.edu/hbooks/pathphys/digestion/smallgut/absorb_aacids .html

What is claimed is:

1. A method of using nanoparticles for contacting a target cell or tissue wherein the nanoparticles are designed to cross the cellular membranes of the target cell comprises:
providing complex nanoparticle structures that specifically resonate or vibrate at least at a specific frequency;
attaching glutathione as a molecular layer or layers to said nanoparticle structures;
wherein other molecules, therapeutic agents, drugs are added to the molecular layer or layers of said nanoparticles structures;
delivering said complex particle structures into said targeted cells; and
using radiation for releasing at least partially said glutathione, with the option to release other molecules, therapeutic agents or drugs from said complex nanoparticle structures in destination target cell.

2. The method of claim 1 wherein said nanoparticles are gold nanoparticles of narrow size distribution prepared in the absence of any dispersant or surfactant with a diameter of between 0.1 nm-40 nm.

3. The method of claim 1 wherein said nanoparticles are gold nanoparticles of narrow size distribution with a diameter of between 10-20 nm.

4. The method of claim 1 wherein said nanoparticles are of gold or silver.

5. The method of claim 1 wherein said complex nanoparticles are anisotropic particles of rods, shells and/or tubular platelets, or other geometric shapes characterized by a high specific surface area.

6. The method of claim 1 wherein said nanoparticles are gold nanoparticles or gold nanoplatelets.

7. The method of claim 1 wherein said nanoparticles are silver nanoparticles or silver nanoplatelets.

8. The method of claim 1 wherein said nanoparticles are gold and silver nanoparticles mixed in various ratios.

9. The method of claim 1 wherein said nanoparticles are gold plated silver nanoparticles or silver plated gold nanoparticles.

10. The method of claim 1 wherein said nanoparticles are structures which display a surface plasmon band resonance or other resonant characteristic.

11. The method of claim 1 wherein said particle structure designed to resonate at specific frequencies that can be exactly or at least closely matched by an external beam of electromagnetic or other type of radiation

12. The method of claim 1 wherein said nanoparticles have an average size in the range of about 0.1-35 nm, and a size distribution where more than 90 percent are within 10% of the average size.

13. The method of claim 1 wherein the surface layer of nanoparticle structures is comprised mainly of reduced glutathione—GSH.

14. The method of claim 1 wherein said surface layer material comprises a molecule of a sulfur, PEG, phosphorus or amine group or any combination thereof.

15. The method of claim 1 wherein said surface layer material comprises molecules of one or more prescription or nonprescription drugs or nutritional supplements such as folic acid.

16. The method of claim 1 wherein said surface material comprises molecules of one or more chemotherapeutic drugs.

17. The method of claim 1 wherein the surface monolayer is comprised of a mixture having different proportions of different molecular structures.

18. The method of claim 15 where the molecular structure of the monolayer is comprised of: aminoacids, peptides or polypeptides, proteins, an antibody or a fragment thereof and/or molecules with a sulfhydryl group, a nucleic acid, a vitamin or enzyme, a carbohydrate molecule, a lipid molecule, a drug, or synthetic molecule or polymer or any combination thereof.

19. The method of claim 1 wherein the step of delivering said particle structures to target cell or tissue which include living cells and tissue of an organism is by administering a specific amount of particle structures which achieves a targeted concentration of said particle structures in said tissue or said population of cells.

20. The method of claim 1 wherein attaching said molecular layers to the complex nanoparticle structures are designed for a specific, cellular/tissue penetration, loading time, stability, half life, elimination pattern, release pattern at the target site or any combination thereof.

21. The method of claim 20 wherein said molecular layers components of the complex nanoparticle structures are designed for a specific pattern of time release at the target site as a result of vibrating at a specific frequency with patterns that are adjusted to be variable or constant patterns of time release ranging from total release, to massive, partial, minimal, or no release at all during a desired period of time with the option to release in a same or different pattern at subsequent periods of time.

22. The method of claim 19 wherein said tissue or said population of cells is a tumor including cancer tumor.

23. The method of claim 19 wherein said organism can be a human organism, animal, plant, bacteria, virus or insect.

24. The method of claim 1 wherein the step of delivering said particle structures to living tissues can be administered orally, intravenously, intra-arterially, transdermally, encapsulated in a liposome, locally or by injection into a body area or cavity.

25. The method of claim 1 wherein delivering said particle structures to living tissues can be administered orally by itself, mixed with a nutritional supplement, attached to a drug or any combination thereof

26. The method of claim 1 wherein delivering said particle structures to living tissues can be administered through implantation of a device capable of slow release of said particle structures ,

27. The method of claim 1 wherein delivering said particle structures to living tissues is administered for achieving specific loading and concentration or concentration gradient of the particle structures in and around the target tissue or tissues of interest

28. The method of claim 1, wherein releasing the molecular layers or monolayer from the complex nanoparticles at the target site destination or vibrating the nanoparticles without releasing a molecular layer is accomplished by exposing the target tissue to a laser, ultrasound or other radiation.

29. The method of claim 1, wherein releasing the monolayer at the target site destination is accomplished by a chemical means such as pH changes

30. The method of claim 28 wherein radiation consist of ultrasound, magnetic fields, electric fields, coherent laser beams, visible light, filtered light or any combination thereof.

31. The method of claim 28 wherein the electromagnetic methods comprise exposing the target tissues to a coherent laser beam with one specific wavelength or a frequency band that corresponds exactly to the plasmon band resonance of the structured nanoparticles, thus triggering specific vibrational resonance of said particles.

32. The method of claim 31 wherein the electromagnetic methods comprise exposing the target tissues to a coherent laser beam with multiple specific wavelengths corresponding exactly to the multiple plasmon band resonances of the structured nanoparticles (eg. Platelets), thus triggering specific vibrational resonance of said particles.

33. The method of claim 28 wherein the target tissues are exposed to continuous, variable or pulsed radiation

34. The method of claim 28 wherein said radiation is in the form of UV-VIZ laser or light beams with a frequency range of between 510 nm for spherical gold particles of 3 nm to a frequency range of 522 nm for spherical gold particles with a diameter of 35 nm.

35. The method of claim 28 wherein said radiation is in the form of UV-VIZ laser beams with a range of about 200-800nm.

36. The method of claim 28 wherein said radiation is in the form of infrared radiation is of wavelengths from 800 nm to 100 micrometers.

37. The method of claim 28 wherein said radiation is in the form of ultraviolet radiation with wavelengths from 10 nm to 400 nm.

38. The method of claim 28 wherein said radiation is in the form of XRAY radiation with wavelengths from 0.01 to 10 nm.

39. The method of claim 28 wherein said nanoparticles absorb said radiation as a surface plasmon band resonance on a wider range of wave length of up to plus or minus 100 nm or more.

40. The method of claim 28 wherein said nanoparticles resonate or entrain with the frequency or frequencies of the external radiation or stimulus having one or more specific wavelength, amplitude, scatter, spin, constructive or destructive interference, squeezing, polarization, coherence or any combination thereof.

41. The method of claim 28 wherein said nanoparticles resonate with the frequency of the external radiation in a specific and consistent way called a surface plasmon band resonance which is dependent of the particle size, structure, shape, and adsorbed species on the surface of the nanoparticle.

42. The method of claim 28 wherein said nanoparticles are purposely manufactured with a particular specific size, shape, structure and adsorbed species on the surface as to resonate or entrain to a very specific frequency

43. The method of claim 28 wherein said nanoparticles are purposely manufactured with a particular specific size, shape, structure and adsorbed species on the surface as to resonate or entrain to a very specific frequency and then directing said specific frequency to the target area to accomplish the resonant vibration of the target particle structure.

44. The method of claim 28 wherein the resonant vibration of the target particle structure is controlled in order to be translated in controlled degrees of localized heat that are designed to speed up specific chemical and enzymatic reactions

45. The method of claim 28 wherein the resonant vibration of the target particle structure is controlled in order to be translated into controlled destructive localized heat that will microscopically or macroscopically destroy surrounding structures in a controlled or uncontrolled fashion.

46. The method of claim 28 wherein the resonant vibration of the target particle structure is translated in partially or completely releasing the monolayer to the target destination.

47. The method of claim 28 wherein the resonant vibration of the target particle structure is designed to accomplish a controlled gradual release of the monolayer.

48. The method of claim 28 wherein the resonant vibration of the target particle structure is designed to release antioxidants to the targeted tissue, living cells and/or intracellular structures.

49. The method of claim 28 wherein the resonant vibration of the target particle structure is designed to release antioxidants, prescription or nonprescription drugs to the mitochondria/cellular structure.

50. The method of claim 28 wherein the resonant vibration of the target particle structure is designed to release glutathione to the cellular structure/mitochondria.
